(19) United States
(12) Patent Application Publication
(10) Pub. No.: US 2021/0325361 A1
(43) Pub. Date: Oct. 21, 2021

(54) System for Indicating Content within a Fluid Container

(71) Applicant: Adam Everett French, Princeton, KY (US)

(72) Inventor: Adam Everett French, Princeton, KY (US)

(21) Appl. No.: 17/231,996

(22) Filed: Apr. 15, 2021

Related U.S. Application Data
(60) Provisional application No. 63/010,488, filed on Apr. 15, 2020.

(51) Int. Cl. G01N 33/28; G01N 21/78; G01N 21/03

(52) U.S. Cl. CPC G01N 33/2841; G01N 2021/7796; G01N 21/03; G01N 21/783

(57) ABSTRACT

A system for indicating content within a fluid container provides an improved fuel container for mixed fuel, which indicates its contents to a user. The system communicates whether there is oil, gas, a mixture of the two, or nothing inside the container. When the system is filled with gas only, the color of the can will change to, or otherwise remain, a first color. Similarly, oil alone would be indicated by a unique color. When a user combines oil and gas, the system changes to a new color to let the user know that oil is indeed inside the receptacle of the apparatus. Alternatively, the system may provide a nozzle equipped with the same color-indicating capabilities as the fuel receptacle embodiment. For example, when oil is mixed with gas inside the system, the nozzle may turn blue to ensure that a user is aware of the change of contents.

The current application claims a priority to the U.S. Provisional Patent application Ser. No. 63/010,488 filed on Apr. 15, 2020.

### FIELD OF THE INVENTION

The present invention generally relates to oil and gas products. More specifically, the system for indicating content within a fluid container provides an improved mix gas can which communicates its contents with a multi-colored chromatic indicator.

### BACKGROUND OF THE INVENTION

Since the industrial era, chemical combustion of gasoline, oil, and petroleum products has been utilized extensively to power machines and devices. These improvements once revolutionized the world, allowing for mass production of goods and the development of new categories of jobs. While other energy sources are becoming more prevalent today, gas and oil are still used both separately and in combination to power many types of heavy machinery.

However, such devices often have particular requirements for their fuel intake. Specific mixtures of gasoline and oil are necessary to prevent engines from becoming damaged due to overheating, locking up, or otherwise failing. It is common for a user of such devices to accidentally fill up machinery with oil or gasoline that does not meet the appropriate mixture specifications for the device being fueled. Such an incident is often caused by utilization of fuel from canisters or containers that do not provide up-to-date information about their contents. What is needed is a device capable of storing liquid oil, gasoline, and combinations thereof without reacting chemically with those contents. Further desirable is a container that indicates its contents during storage.

The present invention addresses these issues. An objective of the system for indicating content within a fluid container is to provide an improved fuel container for mixed fuel, which indicates its contents to a user. The apparatus will communicate whether there is oil, gas, a mixture of the two, or nothing inside the canister. When the apparatus is filled with gas only, the color of the can will change to, or otherwise remain, a first color. Similarly, oil alone would be indicated by a unique color. When a user combines oil and gas, the apparatus changes to a new color to let the user know that oil is indeed inside the receptacle of the apparatus. Alternatively, the apparatus may provide a nozzle equipped with the same color-indicating capabilities as the fuel receptacle embodiment. For example, when oil is mixed with gas inside the apparatus, the nozzle may turn blue to ensure that a user is aware of the change of contents.

### BRIEF DESCRIPTION OF THE DRAWINGS

FIG. 1 is a schematic diagram representing the present invention with a base opaque color indicating that the light-permeable hollow body is empty.

FIG. 2 is a schematic diagram representing the present invention with a first opaque color indicating that the light-permeable hollow body is empty.

FIG. 3 is a schematic diagram representing the present invention with a first opaque color indicating that the light-permeable hollow body is full of gasoline.

FIG. 4 is a schematic diagram representing the present invention with a second opaque color indicating that the light-permeable hollow body is full of oil.

FIG. 5 is a schematic diagram representing the present invention with a second opaque color indicating that the light-permeable hollow body is full of a mixture of gasoline and oil.

FIG. 6 is a schematic diagram representing the chromatic indicator of the present invention.

FIG. 7 is a schematic diagram representing the chromatic indicator of the present invention.

FIG. 8 is a schematic diagram representing the chromatic indicator of the present invention.

### DETAILED DESCRIPTION OF THE INVENTION

All illustrations of the drawings are for the purpose of describing selected versions of the present invention and are not intended to limit the scope of the present invention.

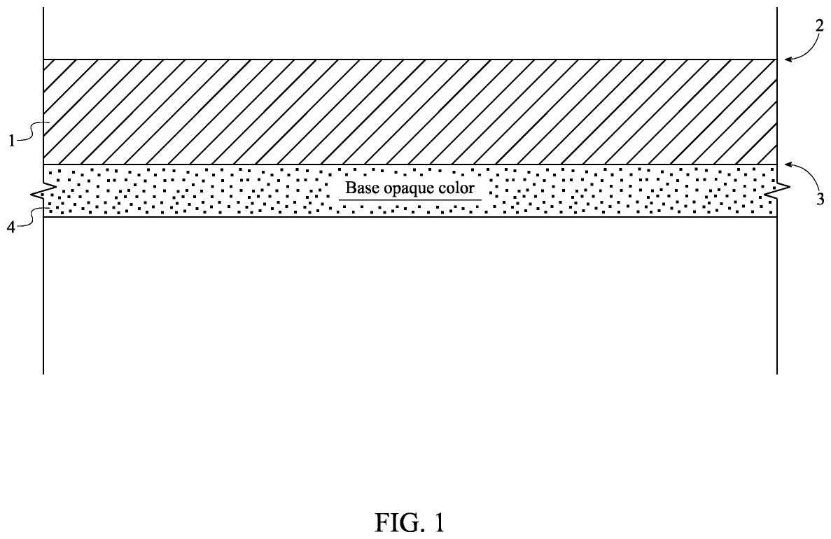

The present invention is a system for indicating content within a fluid container that is used to contain liquid fuel for various combustion-powered tools or machinery. The present invention is also configured to change color to indicate the contents of the fluid container. The present invention comprises a light-permeable hollow body 1 and a chromatic indicator 4, as represented in FIG. 1. The light-permeable hollow body 1 is a chemically-inert receptacle capable of storing varying volumes of concentrated or diluted liquid fuels, including, but not limited to, gasoline, oil, combinations thereof, and more. The chromatic indicator 4 relates to a unit which reacts, preferably chemically, to the presence of various fluids by changing color. The chromatic indicator 4 may alternatively react due to any or any combination of changes in pressure, viscosity of contained fluid, acidity, weight, light dispersion, heat, or a variety of other stimuli and capable of distinguishing different fluids.

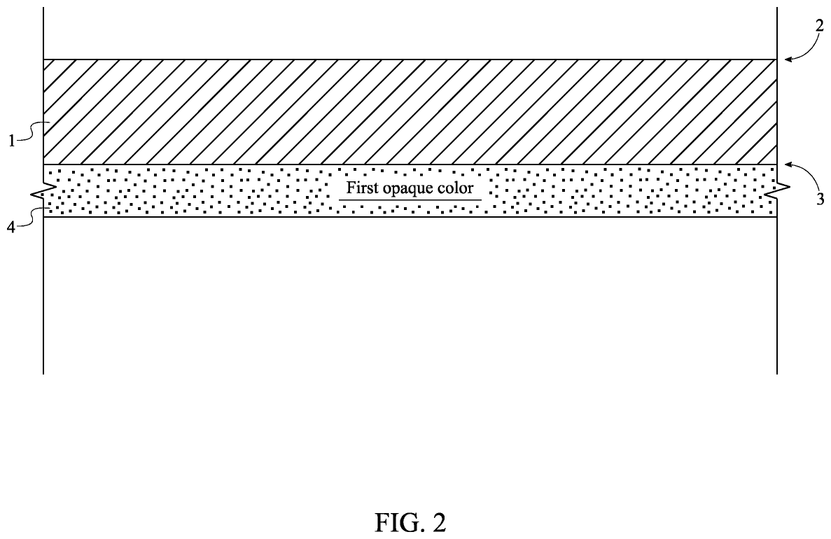

The general configuration of the aforementioned components allows the present invention to efficiently and effectively contain fluids and indicate the content or chemical composition of the fluids. The light-permeable hollow body 1 comprises an outer body surface 2 and an inner body surface 3, as represented in FIG. 2. The outer body surface 2 relates to the face of the light-permeable hollow body 1 that is oriented away from the inside of the light-permeable hollow body 1. Conversely, the inner body surface 3 relates to the face of the light-permeable hollow body 1 that is oriented towards the inside of the light-permeable hollow body 1. The chromatic indicator 4 is superimposed across the inner body surface 3. This arrangement enables a user to view the chromatic indicator 4 as the chromatic indicator 4 changes color due to varying contents of the light-permeable hollow body 1. The chromatic indicator 4 is configured to change to a first opaque color while in physical contact with a quantity of gasoline 5. The first opaque color may be any distinct color on the visible spectrum. Similarly, the chromatic indicator 4 is further configured to change to a second opaque color while in physical contact with a quantity of oil 6. The second opaque color may be any distinct color on the visible spectrum that is separate from the first color. In an exemplary embodiment, the first opaque color and the second opaque color are primary colors. Such an embodiment ensures that the presence of both gasoline and oil is reflected in the chromatic indicator 4 as a distinct secondary color.

The light-permeable hollow body 1 benefits from visual properties that allow the chromatic indicator 4 to be viewed while also preventing excessive light exposure into the contents of the light-permeable hollow body 1. To this end, the light-permeable hollow body 1 may be made of a translucent material. This arrangement ensures that the chromatic indicator 4 can be used to determine the contents of the light-permeable hollow body 1 while the contents are simultaneously protected from degradation due to excessive or prolonged exposure to sunlight.

Intuitive usage of the light-permeable hollow body 1 may alternatively benefit from visual properties that allow the contents of the light-permeable hollow body 1 to be viewed directly. To this end, the light-permeable hollow body 1 may be made of a transparent material. This arrangement allows the contents of the light-permeable hollow body 1 to be viewed and assessed directly through observation of both the chromatic indicator 4 and the light-permeable hollow body 1 itself.

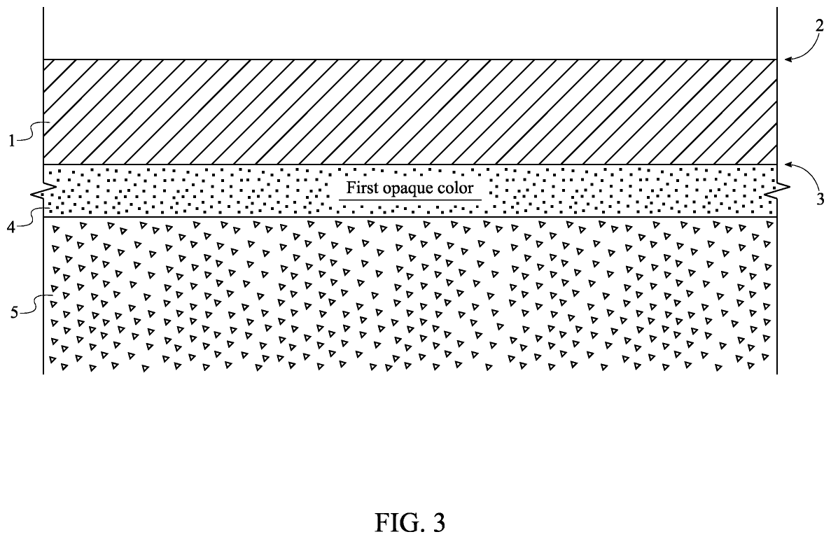

The present invention must be equipped to respond to the presence of gasoline, as gasoline is a common liquid fuel source for combustion machinery. To this end, the chromatic indicator 4 may be compositionally configured to trigger a first non-volatile chemical reaction between the chromatic indicator 4 and the quantity of gasoline 5, and wherein the first non-volatile chemical reaction results in the chromatic indicator 4 changing to the first opaque color, as represented in FIG. 3. The first non-volatile chemical reaction denotes a chemical process by which spectral changes are triggered in the chromatic indicator 4. This configuration ensures that the present invention is equipped to respond appropriately to the presence or absence of gasoline.

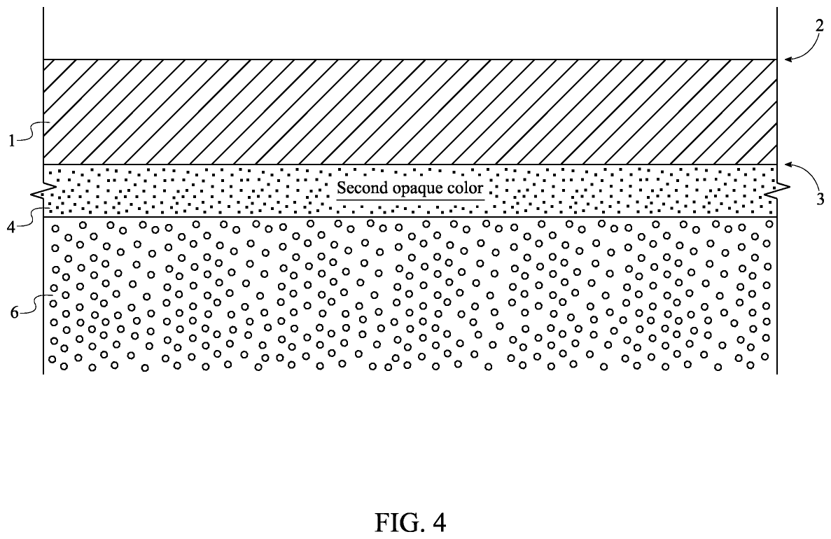

Furthermore, the present invention must be equipped to respond to the presence of oil, as oil is a common liquid fuel source for combustion machinery. To this end, the chromatic indicator 4 may be compositionally configured to trigger a second non-volatile chemical reaction between the chromatic indicator 4 and the quantity of oil 6, and wherein the second non-volatile chemical reaction results in the chromatic indicator 4 changing to the second opaque color, as represented in FIG. 4. The second non-volatile chemical reaction denotes a chemical process by which spectral changes are triggered in the chromatic indicator 4. This configuration ensures that the present invention is equipped to respond appropriately to the presence or absence of oil.

The chromatic indicator 4 benefits further from displaying the presence of gasoline as a primary color, as such a display ensures that the presence of both gasoline and oil results in display of a third distinct color. To this end, the first opaque color may be red. In this way, the presence of gasoline may be clearly indicated.

Often in conjunction with the first opaque color, the chromatic indicator 4 further benefits from displaying the presence of oil as a basic common color, as such a display ensures that the presence of both gasoline and oil results in display of a third distinct color. To this end, the second opaque color may be white. In this way, the presence of oil may be clearly indicated, while the presence of both gasoline and oil may produce a distinctly pink color.

Furthermore, the chromatic indicator 4 may benefit further from displaying the presence of oil as a primary color, as such a display ensures that the presence of both gasoline and oil results in display of a third distinct color. To this end, the second opaque color may be blue. In this way, the presence of oil may be clearly indicated, while the presence of both gasoline and oil may produce a distinctly purple color.

When not in contact with fluids, the present invention may display a color that is distinct from the first opaque color and the second opaque color. To this end, the chromatic indicator 4 may be further configured to remain a base opaque color while in no physical contact with a petroleum-based fluid, as represented in FIG. 1. Thus, the chromatic indicator 4 may visually communicate an absence of fluid within the light-permeable hollow body 1.

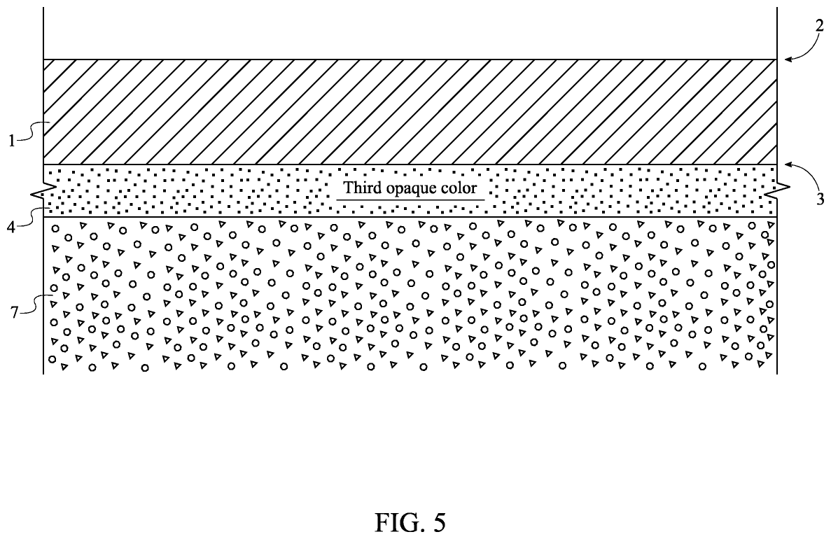

It may further be desirable for the present invention to display a third color, which results in a display that is visually distinct from the first opaque color and the second opaque color. To this end, the chromatic indicator 4 may be further configured to change to a third opaque color while in physical contact with a mixture of gasoline and oil 7, as represented in FIG. 5. Thus, the chromatic indicator 4 may visually communicate the presence of both gasoline and oil within the light-permeable hollow body 1. The third opaque color may shift to a color that is between the wavelengths of the first opaque color and the second opaque color, resulting in a color which may be interpreted as representing a proportion of oil and gasoline based on how close or distant the third opaque color appears to be from the first opaque color and the second opaque color.

Furthermore, the third opaque color may be optimally displayed as a color that is unrelated to the first opaque color and the second opaque color. To this end, the chromatic indicator 4 may be compositionally configured to trigger a third non-volatile chemical reaction between the chromatic indicator 4 and the mixture of gasoline and oil 7, and wherein the third non-volatile chemical reaction results in the chromatic indicator 4 changing to the third opaque color. In this way, the third opaque color may be visually distinct and independent from both the first opaque color and the second opaque color.

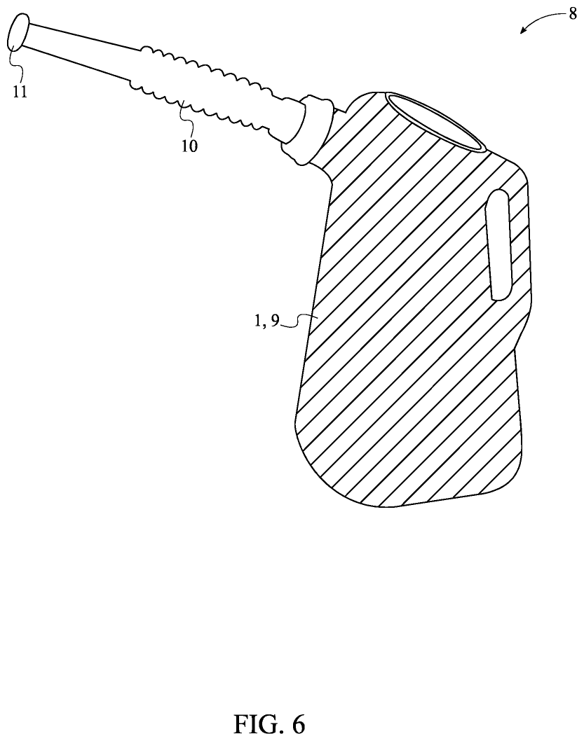

The light-permeable hollow body 1 upon which the chromatic indicator 4 is located may represent a segment of the present invention that does not include the entirety of the fluid retainment section. To this end, the light-permeable hollow body 1 may be a receptacle portion 9 of a fluid container 8, as represented in FIG. 6. This arrangement enables a user to view the contents of the fluid container 8 either through or around the light-permeable hollow body 1, depending on the transparency or translucency of the light-permeable hollow body 1.

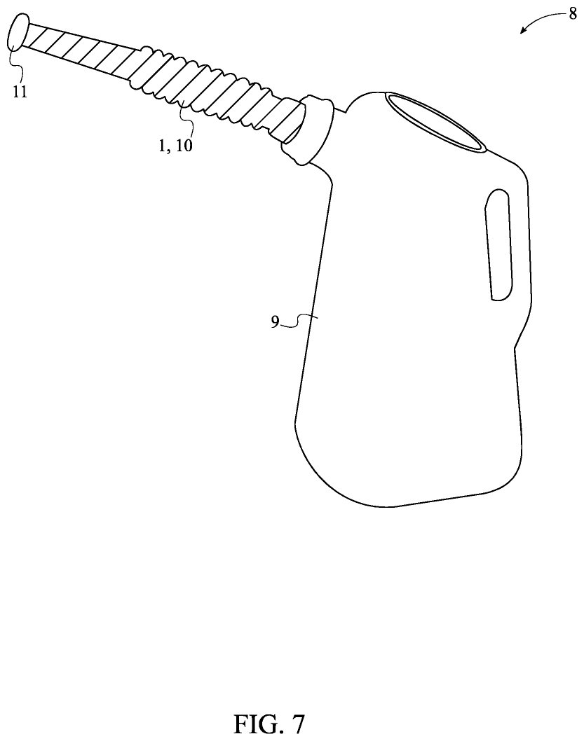

It may further be desirable for the present invention to communicate its contents from another location. To this end, the light-permeable hollow body 1 may be a nozzle portion 10 of a fluid container 8, as represented in FIG. 7. Thus, the user may be made aware of the contents of the fluid container 8 by observing the color of the nozzle.

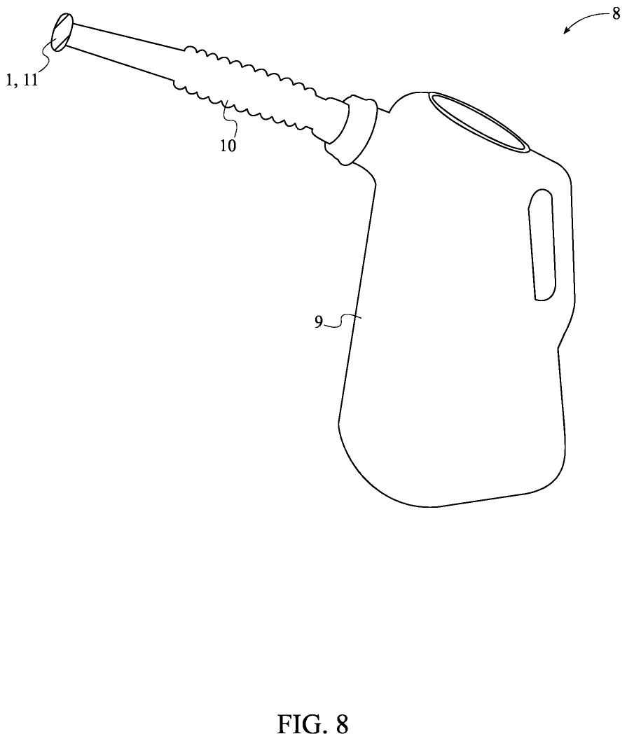

Furthermore, the present invention may best communicate its contents from another alternative location. To this end, the light-permeable hollow body 1 may be a cap portion 11 of a fluid container 8, as represented in FIG. 8. Thus, the user may be made aware of the contents of the fluid container 8 by observing the color of the cap portion 11.

Although the invention has been explained in relation to its preferred embodiment, it is to be understood that many other possible modifications and variations can be made without departing from the spirit and scope of the invention as hereinafter claimed.

What is claimed is:

1. A system for indicating content within a fluid container comprising:
a light-permeable hollow body;
a chromatic indicator;
the light-permeable hollow body comprising an outer body surface and an inner body surface;
the chromatic indicator being superimposed across the inner body surface;
the chromatic indicator being configured to change to a first opaque color while in physical contact with a quantity of gasoline; and
the chromatic indicator being further configured to change to a second opaque color while in physical contact with a quantity of oil.

2. The system for indicating content within a fluid container as claimed in claim 1, wherein the light-permeable hollow body is made of a translucent material.

3. The system for indicating content within a fluid container as claimed in claim 1, wherein the light-permeable hollow body is made of a transparent material.

4. The system for indicating content within a fluid container as claimed in claim 1, wherein the chromatic indicator is compositionally configured to trigger a first non-volatile chemical reaction between the chromatic indicator and the quantity of gasoline, and wherein the first non-volatile chemical reaction results in the chromatic indicator changing to the first opaque color.

5. The system for indicating content within a fluid container as claimed in claim 1, wherein the chromatic indicator is compositionally configured to trigger a second non-volatile chemical reaction between the chromatic indicator and the quantity of oil, and wherein the second non-volatile chemical reaction results in the chromatic indicator changing to the second opaque color.

6. The system for indicating content within a fluid container as claimed in claim 1, wherein the first opaque color is red.

7. The system for indicating content within a fluid container as claimed in claim 1, wherein the second opaque color is white.

8. The system for indicating content within a fluid container as claimed in claim 1, wherein the second opaque color is blue.

9. The system for indicating content within a fluid container as claimed in claim 1, wherein the chromatic indicator is further configured to remain a base opaque color while in no physical contact with a petroleum-based fluid.

10. The system for indicating content within a fluid container as claimed in claim 1, wherein the chromatic indicator is further configured to remain the first opaque color while in no physical contact with a petroleum-based fluid.

11. The system for indicating content within a fluid container as claimed in claim 1, wherein the chromatic indicator is further configured to change to a third opaque color while in physical contact with a mixture of gasoline and oil.

12. The system for indicating content within a fluid container as claimed in claim 11, wherein the chromatic indicator is compositionally configured to trigger a third non-volatile chemical reaction between the chromatic indicator and the mixture of gasoline and oil, and wherein the third non-volatile chemical reaction results in the chromatic indicator changing to the third opaque color.

13. The system for indicating content within a fluid container as claimed in claim 1, wherein the light-permeable hollow body is a receptacle portion of a fluid container.

14. The system for indicating content within a fluid container as claimed in claim 1, wherein the light-permeable hollow body is a nozzle portion of a fluid container.

15. The system for indicating content within a fluid container as claimed in claim 1, wherein the light-permeable hollow body is a cap portion of a fluid container.
